(19) United States
(12) Patent Application Publication
(10) Pub. No.: US 2024/0382707 A1
(43) Pub. Date: Nov. 21, 2024

(54) Bidirectional Spirometer T-Piece

(71) Applicants: George Reed, Lake Oswego, OR (US); Palrick Boileau, Cornelius, OR (US); Wessam Mohamed

(72) Inventors: George Reed, Lake Oswego, OR (US); Palrick Boileau, Cornelius, OR (US); Wessam Mohamed

(21) Appl. No.: 18/786,198

(22) Filed: Jul. 26, 2024

Related U.S. Application Data
(63) Continuation-in-part of application No. 17/335,434, filed on Jun. 1, 2021.

(51) Int. Cl. A61M 16/00; G16H 20/30

(52) U.S. Cl. CPC A61M 16/024; A61M 16/0003; A61M 16/0051; G16H 20/30; A61M 2016/0027; A61M 2205/18; A61M 2205/3327; A61M 2205/3331; A61M 2205/3561; A61M 2205/3592; A61M 2205/581; A61M 2205/583; A61M 2205/8206

(57) ABSTRACT

A respiratory therapy device that is adapted to matingly, frictionally connect with dimensionally standardized devices and device adaptors, that when combined, allow for respiratory treatments. It monitors the direction of the air and or medicament flow (gases) in the device and can visually report as well as record the number of PEP treatment events or the number of medicament administrations by utilizing at least one bidirectional motion sensor therein. It presents a visual and optional auditory alert when the patient's respiratory treatment is not adhered to within waking hours.

### CROSS REFERENCE TO RELATED APPLICATIONS

This application incorporates in its entirety, and claims the benefit of priority from pending U.S. Utility patent application Ser. No. 17/335,434 filed Jun. 1, 2021 entitled “BIDIRECTIONAL INCENTIVE SPIROMETER” which claims priority from U.S. Utility patent application Ser. No. 16/162,343 filed Oct. 16, 2018 (issued as U.S. Pat. No. 11,045,112 on Jun. 29, 2021) and entitled “INCENTIVE SPIROMETER” which claims domestic priority from abandoned U.S. Utility patent application Ser. No. 14/938,805 filed Nov. 11, 2015 and entitled “RESPIRATORY MEDICAMENT AND THERAPY DATA SYSTEM AND METHOD OF USE.”

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to medical respiratory devices, and more particularly to respiratory patient spirometer technology.

### BACKGROUND

Respiratory treatments vary considerably as do the different manufacturer's devices used to facilitate the treatments. Generally, the patient undergoes separate regimens of positive expiratory pressure (PEP) treatments and medicament inhalation (drug delivery). First, the exhalation treatment requires the patient to breathe through any of a plethora of devices that work to increase lung mechanics and or prevent atelectasis. Incentive spirometers (IS) encouraging deep breathing. PEP devices increase pressure in the lung to help keep airways open, and oscillating positive expiratory pressure (OPEP) devices send a resultant pulsation pressure wave back down into the lungs to mobilize mucus and secretions. Second, once enough cycles have been performed, the patient's breathing ability is improved and they can undergo an inhalation treatment wherein they receive a medicated aerosol (generally inhaled antibiotics, bronchodilators, corticosteroids) to further increase their lung capacity and ease their labored breathing. This type of drug delivery is most effective immediately after the exhalation treatments are performed. Switching between devices to accomplish these two tasks, often requires connection to multiple transition components and mouthpieces, which increases the connection complexity and robs precious time between treatments.

Keeping track of the actual numbers, duration and length of the respiratory treatments is an important factor in determining further treatment regimens. Generally, it requires some level of medical staff, be it a doctor, nurse or technician, to both ensure the treatments are performed to satisfaction and on the correct schedule. Knowing this, along with the patient's progress, allows the medical staff to alter the treatment plan accordingly. This information is indispensable but often not available where the trained patient self-administers.

Prior art devices that have tried to combine the two types of respiratory therapies have failed in maintaining a high percentage of the flow of the medicinal aerosol particles in the desirable 0.5 to 4.5-micron diameter. Some of the larger aerosol particles are lost in the exhalation phase where they are swirled around in the device to collide and condense. In devices that utilize a valve system, aerosol particles collide with any valve therein as it opens in the inhalation therapy phase. All these mechanisms reduce the amount of medicated aerosol particles that are delivered to the patient and increase the average size of the aerosol particles delivered. A straight line Laminar gas flow is the optimal route for intake or expulsion of gases to minimize friction, particle impingement and condensation of the medicament in the related equipment.

There are other inherent problems with positive exhalation pressured devices. These are quite exhausting for many patients and because of their diminished lung capacity, successful exhalations (events) often leaves them out of breath and gasping for air. Often the patient does not exhale with enough pressure to achieve the desired effect of loosening the phlegm and opening the pulmonary pathways for inhaled drug delivery. In this case the delivered drug does will be inadequate and the patient's progress will be slower. Unfortunately, with conventional devices the medical staff as well as the patient have no way of knowing what part of the treatment, the PEP or drug delivery, is responsible for the slow progress.

One of the problems with either the administration of an inhalation drug or the performance of a lung-clearing device is because of their frequent usage, they are usually performed unsupervised, by the patient alone. Patients often do not follow the prescribed routines and often do not perform the correct number of events or perform the events correctly. Often the patient has some level of dementia or Alzheimer's and does not know they have forgotten to perform their routine treatments. If medical personnel are not present to monitor the adherence to the patient's treatment schedule, the desired results are not accomplished.

A novel design for a respiratory interface or intermediary apparatus, capable of connection with a diversity of respiratory devices and that provides efficiency in treatment compliance, product delivery and data recording, would fulfill a long felt need in the respiratory treatment industry. This new invention utilizes and combines known and new technologies in a unique and novel configuration to overcome the aforementioned problems and accomplish this.

### BRIEF SUMMARY

In accordance with various embodiments, an improved bidirectional incentive spirometer T-piece that uses at least one capacitive, pressure differential air flow sensor to detect and monitor flow/usage, and can visually and or audibly alert the patient if a scheduled therapy session has been missed during waking hours, so as to encourage them to practice the actions prescribed to keep their lungs healthy, is provided.

In one aspect, a device that provides a visual and optional audible stimulus for the performance of the prescribed pulmonary treatment regime and creates an optional data record of the direction, duration, pressure and number of gas pulse flow events (medicament inhalation or PEP exhalation) that occur within the device during a lung-clearing and treatment pulmonary regime, is provided.

In another aspect, a device that algorithmically analyzes the gas pulse flow data so as to provide a data record of patient treatment that can be appended to their medical history for evaluation against their treatment progress by medical personnel is provided.

In yet a final aspect, a liner, bidirectional incentive spirometer T-piece that enables a new level of efficiency in charting patient progress, medicament delivery, patient follow-up, therapy session data and the like that is adapted to matingly connect with all standardized respiratory devices commonly utilized in all lung and respiratory disease treatment, testing, rehabilitation, medicament delivery and life support devices.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a right side perspective view of the bidirectional spirometer T-piece;

FIG. 2 is a right side view of the bidirectional spirometer T-piece;

FIG. 3 is a top view of the bidirectional spirometer T-piece;

FIG. 4 is a distal end view of the bidirectional spirometer T-piece;

FIG. 5 is a left side of the bidirectional spirometer T-piece;

FIG. 6 is a bottom view of the bidirectional spirometer T-piece;

FIG. 7 is a left side cross-sectional view of the bidirectional incentive spirometer;

FIG. 8 is a right side cross-sectional view of the bidirectional incentive spirometer;

FIG. 9 is an enlarged view of region A of FIG. 8;

FIG. 10 is a representative view of the inhalation sensor in operation during a medicinal inhalation treatment; and

FIG. 11 is a representative view of the exhalation sensor in operation during a positive exhalation exercise.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention. It will be apparent to one skilled in the art, however, that other embodiments of the present invention may be practiced without some of the disclosed details. It should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers herein used to express quantities, dimensions, and so forth, should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

It will be understood that when an element or layer is referred to as being “on,” “coupled to,” or “connected to” another element or layer, it can be directly on, directly coupled to or directly connected to the other element or layer, or intervening elements or layers may be present. In contrast, when an element is referred to as being “directly on,” “directly coupled to,” or “directly connected to” another element or layer, there are no intervening elements or layers present. Like numbers refer to like elements throughout. As used herein, the term “and/or” includes any and all combinations of one or more of the associated listed items.

The terminology used in the description of the bidirectional incentive spirometer herein is for the purpose of describing particular embodiments only and is not intended to be limiting of the inventive concept. As used in the description of the bidirectional incentive spirometer and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “PEP” or “PEP device” refers to a positive expiratory pressure device that provides resistance to the exhalation of breath. This forces respiratory patients to exhale hard into it, increasing the time to empty the lung's capacity against the resistance. This sends a back pressure down the patient's lungs and acts as an airway clearance technique to help air get behind the mucus to move it from lung and airway walls. It also holds airways open, keeping them from closing. There are oscillating and non-oscillating PEP devices of varying designs.

As used herein, the term “delivered drug dose” refers to the aggregate amount of aerosol medicament determined to have reached the patient's lungs in a single drug delivery treatment/session. This is can be roughly calculated knowing how many breaths the patient took, the duration of the breaths, the concentration of the aerosol and the efficiency of the drug delivery device considering the aerosol attenuation.

As used herein, the term “inhalation drug delivery device” refers to any one of a group devices that disperse an aerosol or powder pulmonary medicine. These come in various designs such as nebulizers, pressurized metered-dose inhaler (pMDI), and dry powder inhalers (DPIs).

As used herein, the terms “microprocessor, microcontroller and Application Specific Integrated Circuits (ASICs)” are used interchangeably and all mean a integrated circuit on a microchip that contains all, or most of, the central processing unit (CPU) functions and is the “engine” that goes into motion when the pressure sensing unit detects flow (via a pressure differential across its capacitive differential pressure sensor/s). It incorporates a real time clock and either or both of volatile/nonvolatile memory and performs algorithmic and logic operations to derive data based on input signals from the sensors. It outputs operational signals that integrates with other electrical circuits. It may also output algorithmically derived data to an external computing device. (This may be a local computer, tablet, smart phone, or a health provider's network via a remote server.) These operations are the result of a set of instructions that are part of the device's design as is well known in the industry. In simple terms, these devices are each a multipurpose, programmable device that accepts digital data as input, processes it according to instructions stored in its memory, and provides results or initiates actions such as turning on a LED or initiating an audible alert as output. They may have an integrated wireless transmitter/transceiver for wireless transmission of data (WiFi, near field communication, RF, etc.) directly or indirectly (via intermediary data storage and transfer devices such as hard drives, memory stick, thumb drive, data chip and the equivalent) to an external computing device. They may also be configured to utilize a hard wired data port such as a USB port or a pin system for their direct or indirect data transfer.

As used herein, the term “pressure sensing unit” refers to an application specific integrated circuit (ASIC), microprocessor or microcontroller on a printed circuit board, integrated with at least one capacitive pressure differential air flow sensor, a battery, an LED and an optional audible alarm (piezoelectric buzzer). Optionally, the microprocessor has a wireless transceiver or data transmission port. Herein in general terms, it is an electromechanical device that provides an electronic signal to a microprocessor, microcontroller, or ASIC upon movement of a foil on a moveable plate with respect to its distance from a fixed plate in its capacitive differential pressure sensor/s. The microprocessor algorithmically interprets these changing capacitive signals to affect and transmit data accumulation and to operate visual or audible alerts.

As used herein, the term “capacitive pressure differential sensor” refers to an encapsulated device having a sensing element with a moveable first plate with a conductive foil across it held in a spaced insulated configuration from a fixed second plate. One side of the pressure sensor capsule has an internal port that opens into the interior cavity of the body of the device. The opposing side of the pressure sensor capsule has an external port that opens to the ambient atmosphere. The foil stretched across the moveable first plate moves closer to, or further away from, the fixed second plate within the sensor capsule based on the differential pressure across the foil. This change in proximity between the moveable first plate and the fixed second plate changes the capacitance of the circuit as measured by the microprocessor, microcontroller or ASIC. The pressure of the gas pulses (the patient's inhalation or exhalation) passing in either direction along the central cavity of the bidirectional incentive spirometer is either greater than or less than the ambient atmospheric pressure. The sensing element may be directly connected to the microprocessor, microcontroller or ASIC or may have an active or passive transducer connected to the sensing element that translates the motion of the sensing element into an electric signal that provides the direction, duration and optionally the magnitude of the gas pulse flow down the internal cavity of the bidirectional incentive spirometer to the microprocessor, microcontroller or ASIC.

As used herein, the term “transducer” refers to any mechanical, electrical, electronic or electro-mechanical device that either passively or actively translates the mechanical motion or position of a sensing element into an electronic signal that it provides to the microprocessor, microcontroller or ASIC which is the algorithmically converted into and stored as digital data that is reflective of the direction, duration, pressure and time of the occurrence of the gas pulse flow event.

As used herein, the term “personal mobile device” refers to a device that is both portable and capable of collecting, storing, transmitting or processing electronic data or images. Examples include laptops or tablet PCs, personal digital assistants (PDAs), and “mobile smart” phones. This definition also includes storage media, such as USB hard drives or memory sticks, SD or CompactFlash cards, and any peripherals connected to the device.

As used herein, the term “smartphone” means any web-enabled mobile phone. While the term “smartphone” is well known in the art, smartphones typically include a touch sensitive screen, a messaging client, global positioning systems (GPS) technology or any other geo-position mechanisms to determine the physical coordinates of the smartphone, and a browser application. The browser application employs any web-based language such as JavaScript Object Notation (JSON), JavaScript, HyperText Markup Language (HTML), or any other web-based programming language capable of sending and displaying messages, search queries, and search query results.

The present invention relates to a novel design for an improved linear, bidirectional incentive spirometer T-piece 2 that is connectable to various positive exhalation pressure (PEP) devices and medicament inhalation (aerosol drug delivery) devices. It has a real time clock and will have both the time of day as well as the patient's respiratory treatment schedule loaded into its microprocessor's algorithm. If during waking hours the respiratory treatment schedule is not followed, the T-piece will present an onboard visual and/or audible alert. Hopefully, this will spur the patient to resume their treatments. Optionally, it may provide data about the gas flows through the device for review by medical personnel. These data records are transmittable (wirelessly or directly) via an intermediary data transfer device or not, to a remote computing device that has an associated application installed thereon that can download the respiratory data and perform algorithmic analyses thereof.

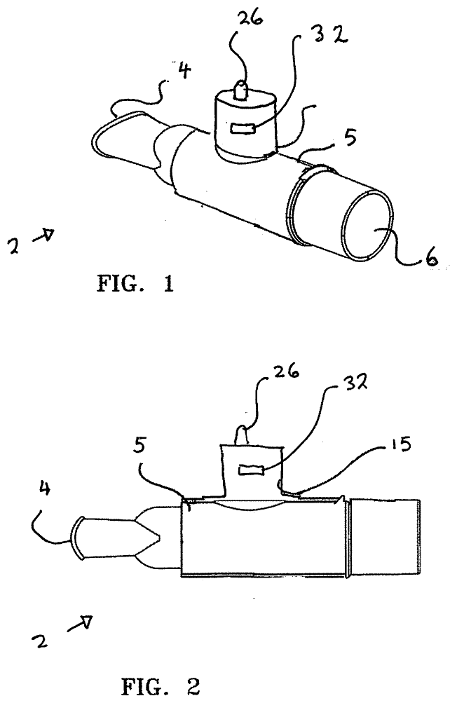

Looking at FIG. 1 it can be seen that the T-piece 2 allows the connection of an airway clearance device (as an example, a “PEP” device), an inhalation drug delivery device (that uses an inhalation to deliver a medical powder or aerosol) or other pulmonary equipment such as a ventilator or respirator or gas supply. The T-piece 2 has a proximal (patient mouthpiece) end 4 and a distal (connected device) end 6 with the same or different IDs and ODs, but of standardized ISO dimensions. In the preferred embodiment the proximal end 4 of the device has a 15 mm ID (female) port and the distal end 6 has a 22 mm OD (male) port.

Since pulmonary equipment have standardized end coupling sizes, generally with ISO 22 mm ID, ISO 22 mm OD, 15 mm ID or 15 mm OD dimensions, there are copious adapter sleeves available in the industry to connect equipment with different sized end couplings. This has the benefit of allowing the quick connection, either directly or indirectly, of the T-piece 2 with an adaptor sleeve, to any of the above devices. Consequently, the T-piece 2 may have the same sized ends or different sized ends, and with the use of reducing or increasing adapter sleeves, can be connected at either the proximal or distal end to any piece of pulmonary equipment.

Currently patients with lung disorders and respiratory ailments undergo testing, medicament delivery, monitoring, phlegm dislodgment therapy, lung expansion therapy and other related medical procedures. Many of these are done in the hospital under medical supervision, while others may be as simple as a nebulizer aerosol medicament session or a PEP regime performed at home. The efficiency of the implementation of these treatments can only be determined analyzing data not generally collected and that possibly may only be orally reported. The overall actual physical results of these treatments are sometimes charted, however there is no ongoing complete record of the performance of each of these treatments for future trending analyses. This is a huge downfall of respiratory disorder treatments.

Because these pulmonary treatments occur so frequently (often a prescribed protocol is every two hours) and usually unsupervised, the overall effect or progress of these treatments is not always discernable, even to those medically trained in the industry. One reason, is that the patient may not follow the treatment schedule, may not complete each gas flow event, or may not be able to generate enough pressure or suction with their breath. Alzheimer's patients frequently need retraining in how and when to conduct their personal home treatments. The inability of the respiratory disease treatment industry to tabulate the actual medicament delivered to a patient's lungs, or how often they actually and correctly used their PEP device is a huge downfall that this linear, bidirectional, positive inhalation spirometer T-piece can remedy.

This linear, bidirectional incentive spirometer T-piece 2, is a T-shaped intermediary device that connects between the patient (the mouthpiece) and the patient's pulmonary treatment equipment and performs two tasks: that of visually and audibly providing a patient alert when a therapy session has been missed, provides respiratory data to reflect the therapy sessions and treatments conducted.

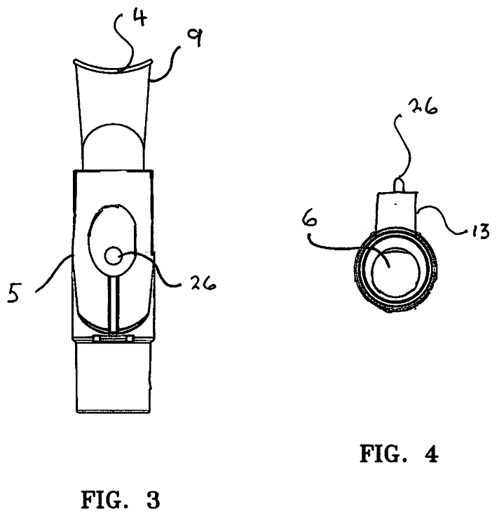

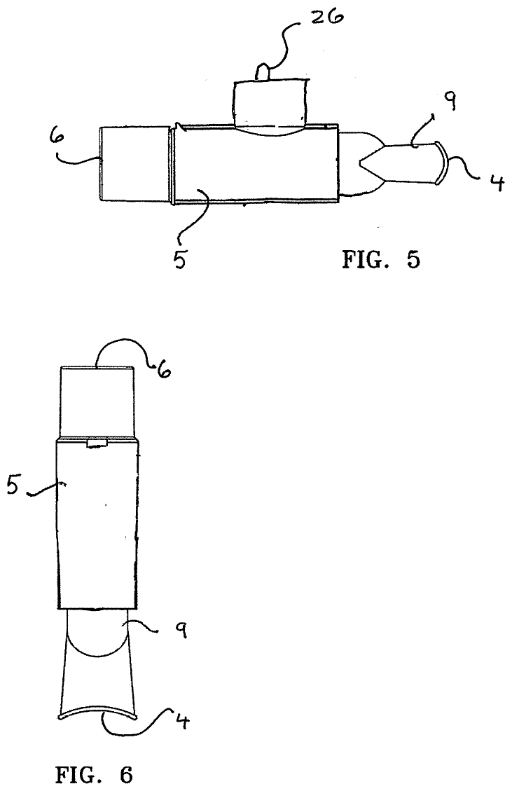

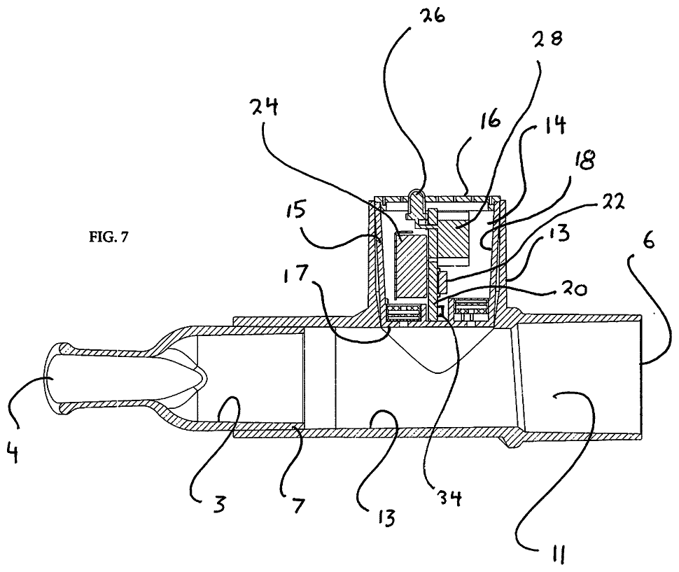

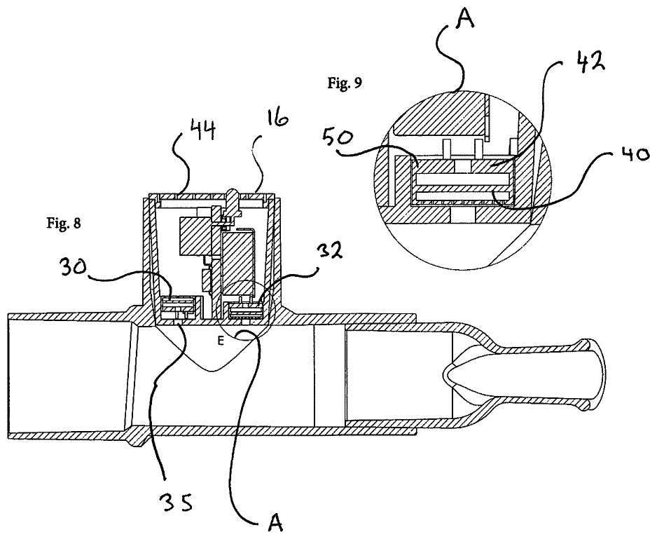

Looking at FIGS. 1 to 9, it can be seen that the incentive spirometer T-piece (T-piece) 2 has a main linear, cylindrical, hollow polymer body 5 with a distal (connected device) end port 6 and a proximal (patient) end port 4. These two ports 4 and 6, reside centered about the linear axis, of the T-piece 2 to allow a straight, laminar flow, minimal resistance pathway for the flow of gas pulses in either direction. On the body 5 of the T-piece 2, between the proximal end port 4 and the distal end port 6, is an open, hollow cylindrical stub 13 extending perpendicularly from the linear axis of the body 5. This stub 13 is tapered inward toward the body 5 and is open on its top end and its bottom end into the central cavity of the body 5. This stub 13 is where the pressure sensing unit 14 in its housing 15 (which is tapered for frictional mating engagement), is affixed.

The internal cavity 11 of the T-piece 2 has a smooth internal face 13 to minimize condensation of moisture in exhaled breath, to minimize the condensation of the atomized medicament and to facilitate the sterilization of the T-piece 2. (See FIG. 7) The minimum Ra or arithmetic average roughness value for the internal face 13 is 8 Ra or 3 microns. The preferred embodiment has a surface roughness range of 3-9 Ra.

The proximal end port 4 is designed to accept a standardized replaceable mouthpiece 3, and in the preferred embodiment is configured as a female ISO 15 mm circular inner diameter port 8. Into this 15 mm port is frictionally inserted a mouthpiece 3 having a matingly engageable male 15 mm OD distal end 7 and an oval proximal end 9 that the patient places in their mouth. Other size matingly engageable configurations work equally as well, however, a 15 mm OD patient mouthpiece is the most prevalent size in the industry. The actual OD and ID may be varied during fabrication or may be adjusted through the use of a reducing or increasing adapter.

The distal end port 6 is designed for connection to a plethora of different pulmonary treatment devices, primarily a PEP device or an inhalation drug delivery device. The preferred embodiment is configured as a tapered male ISO 22 mm circular OD for connection with industry standard nebulizers and similar functioning devices. The outer surface of the distal end port 6 of the body 4 is circular and unadorned. The outer tapered configuration of the distal end port 6 allows for an extra secure connection with any PEP device, which is important because there is a backpressure developed during patient exercises trying to dislodge the PEP device from the T-piece 2. Again, other dimensions of matingly engageable configurations will work equally as well, however, a 22 mm OD is the most prevalent size in the industry.

The pressure sensing unit 14 has a tapered cylindrical housing 15 with a fitted top cap 16 and a bottom plate 17 affixed to its circular side wall 18. In the housing 15 is a printed circuit board (PCB) 20 with a microprocessor 22 mounted thereon. This is operationally connected to a battery 24, an LED 26, a buzzer 28, an encapsulated inhalation sensor 30, an encapsulated exhalation sensor 32. There is an optional hard wire data transfer port 32 (FIGS. 1 and 2) in embodiments where a wireless transceiver 34 is not present on the printed circuit board 20. (FIG. 7) There are orifices 35 in the bottom plate 17 that allow the pressure in the cavity of the body 5 to reach one side of the foil 40.

The pressure sensing unit 14 uses two encapsulated capacitive differential pressure sensing units 30 and 32 which are identical but are mounted inverted from each other. These units exhibit an increase in capacitance (detectable and measurable by the microprocessor) when the foil 40 on the moving plate, sensing a pressure differential between its two sides, moves closer to the fixed plate 42. (See FIG. 9) While the patient inhales 99, the pressure in the body 5 of the T-piece 2 is briefly less than the ambient pressure in the housing 15 (which is open to the atmosphere because of the orifices 44 in the top cap 16 and in the capsule 50.) This lower pressure is transmitted to the underside of the foil 40 (of sensor 30) through either orifices in the fixed plate 42 or through the space between the fixed plate 42 and its capsule 50. The foil 40 is sucked toward the fixed plate 42 and the microprocessor 22 senses and measures the change in capacitance which it uses to algorithmically determine the flow pulse, its duration, and its intensity. The direction of the flow pulse is discernable through the use of a second encapsulated capacitive differential pressure sensing unit 32.

The second, inverted encapsulated capacitive differential pressure sensing unit 32 responds in the same manner except that its foil 40 deflects closer to the fixed plate 42 upon the patient exhaling into the T-piece. The use of the two sensors (inverted with respect to one another) allows the microprocessor to determine which direction the flow of air is moving in. One sensor is designated the inhalation sensor 30 and one sensor is designated the exhalation sensor 32 in the microprocessor's algorithm.

The T-piece's microprocessor's algorithmic program has the patient's treatment regime as well as the time of the day. The algorithm will reference the pulses of air along the cavity of the T-piece as well as the time of day (via its real time clock) so as to alert the patient with the LED and/or audible buzzer when a treatment has been missed within waking hours. The alert is initiated by the microprocessor when the period between respiratory treatments exceeds the preset duration during waking hours. Once a differential pressure is detected again, the T-piece's microprocessor will reset and turn the alert off. In the sleeping hours the unit will power itself down to conserve battery power. In a common scenario, a patient may be assigned PEP exercises every 2 waking hours of the day with a bedtime of 9 PM to 7 AM.

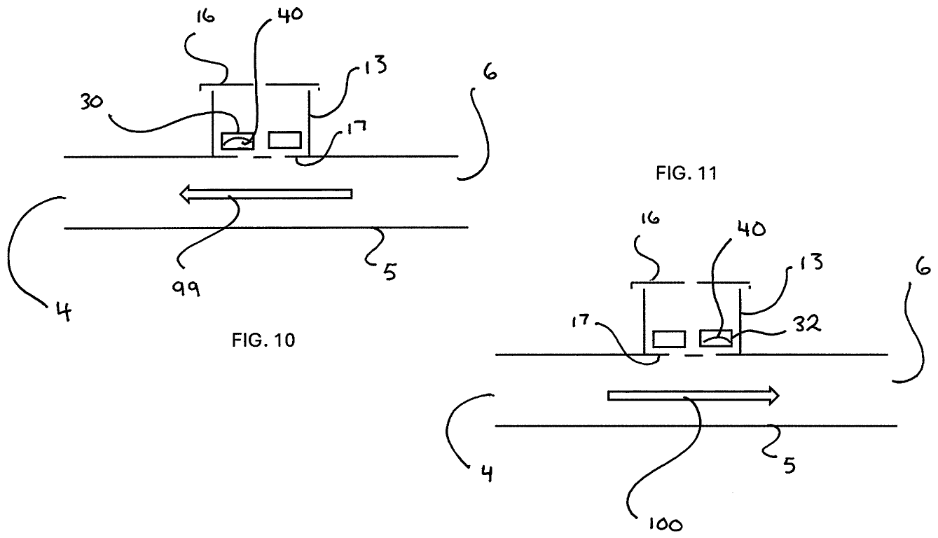

Looking at FIG. 10 an inhalation 99 treatment is illustrated. The direction of flow of the patients inhalation 99 causes a momentary differential in pressure within the capsule of the inhalation sensor 30 that causes the foil 40 to deflect closer to the fixed plate. This causes a change in its capacitance. The micro controller unit on the integrated chip senses this capacitance change and algorithmically determines if an event has occurred, at what time it occurred, which sensor (inhalation 30 or exhalation 32) experienced the capacitive change, and the magnitude and duration of the capacitive change. From these measured variables, the direction, volume, pressure, and adherence to the scheduled treatment may be algorithmically determined.

FIG. 11 illustrates an exhalation 100 treatment. Here the second inverted sensor 32 has its foil 40 deflected. The operation is identical to that described for FIG. 10 except that the capacitance change of the designated exhalation sensor 32 allows the microprocessor to differential the direction of the patient's breath. The microprocessor then can distinguish an exercise from a medicament delivery, and having its own internal time clock records each in the series of these events by date, time and duration.

The microprocessor, now knowing what type of respiratory treatment has occurred can reset the timer for its incentive visual alarm (LED) and/or audible alarm (buzzer) intervals. If the time between respiratory treatments exceeds a preset limit, the patient is given the incentive visual and/or audible alerts, prompting them to attend to their treatment.

In operation, the patient frictionally engages the appropriate respiratory device to the distal end port 6 of the bidirectional spirometer T-piece. (Generally, this will first be a PEP device to first loosen the phlegm from the patient's lungs.) For this PEP phlegm clearing cycle, the microprocessor records the time the inhalation sensor 30 reacts to the gas pulses flowing by through the T-piece, records the direction of flow, the strength and duration of the gas pulses encountered in the cycle. The patient then removes the PEP device and connects the medicament delivery device and begins the drug delivery cycle. The gas pulses are again detected and recorded as in the PEP phlegm clearing cycle. Since the direction of the gas pulses are in the reverse direction as indicated by the sensing element 32, the microprocessor can differentiate whether it is an exercise or a medicament delivery. When finished, the patient disconnects the medicament delivery device. At this time, all of the individual pulmonary events have been recorded, and with the microprocessor not receiving any gas pulse signals for a preset period of time, goes into a low power hibernation mode until the next capacitance change is detected. The technician may insert a memory stick prompting the microprocessor to export all of its data onto the memory stick. Alternately, the technician may use their smart device (cell phone, tablet, computer etc.) to pair to the microprocessor and initiate a data transfer from the microprocessor via an application installed on the smart device.

As described herein, the bidirectional spirometer T-piece 2 allows connection to any PEP device, medicament delivery device or other pulmonary therapy device. Its linear design minimizes the amount of aerosol condensing on the devices inner walls and maximizes the aerosol particle size transmission efficiency. It monitors, records and is able to transmit a record of the patient's pulmonary treatment data. It also provides a patient incentive to perform all their treatments on the schedule set out for them in their waking hours, in the form of a visual or audible alert. The transmitted data may be read by medical personnel who can determine when the lung treatments are correctly, and timely performed. With this information the medical staff can corelate the patient's progress to their performance of phlegm clearing exercises and dosage of aerosol medicament. Therein, more informed medical decisions can be rendered.

The data collected per phlegm clearing cycle or medicament cycle by the microprocessor, allows the medical practitioner to review the patient's progress against their adherence to the pulmonary treatment regime, to calculate the delivered drug dose for adjustment of the patient's medication and to place the patient's pulmonary treatment data into their medical record. This offers a huge step forward in the evaluation and review of the treatment of respiratory ailments.

While certain features and aspects have been described with respect to exemplary embodiments, one skilled in the art will recognize that numerous modifications are possible. Consequently, although several exemplary embodiments are described above, it will be appreciated that the invention is intended to cover all modifications and equivalents within the scope of the following claims.

Having thus described the invention, what is claimed as new and desired to be secured by Letters Patent is as follows:

1. A bidirectional incentive spirometer T-piece for connection to and use with pulmonary treatment and drug delivery devices, comprising:
a T-shaped housing, made of a hollow linear body with a linear axis, having a distal end port, a proximal end port, and an open stub, extending perpendicularly therefrom said body, said housing defining a T-shaped cavity therebetween said distal end port, said proximal end port and a top end port of said stub;
a pressure sensing unit affixed within said cylindrical stub, wherein said pressure sensing unit has a pressure sensing body with a side wall, a top cover and a bottom plate with orifices therethrough, and comprises at least one microprocessor on a printed circuit board operatively connected to at least one pressure sensor, an LED, and a battery therein said pressure sensing body;
wherein said pressure sensor provides a signal to said microprocessor in response to the detection of a pulse flow of gas along said linear axis of passage of said housing between said distal end port and said proximal end port, and said microprocessor algorithmically processes said signal into digital data and determines if a patient's respiratory treatment schedule has been adhered to.

2. The bidirectional incentive spirometer T-piece of claim 1 wherein said at least one pressure sensor is a pair of capacitive differential pressure sensors that are mounted in said pressure sensing body in an inverted configuration with respect to each other.

3. The bidirectional incentive spirometer T-piece of claim 1 further comprising a buzzer connected to said microprocessor to provide an audible alert when a patient's respiratory treatment schedule has not been adhered to.

4. The bidirectional incentive spirometer T-piece of claim 1 wherein said microprocessor has a real time clock and a memory, and algorithmically determines the direction of said pulse flow of gas, which respiratory treatment has been performed and its time of occurrence from said pressure sensor signal, and actuates said LED if said respiratory treatment does not occur on a defined time schedule.

5. The bidirectional incentive spirometer T-piece of claim 4 wherein said microprocessor also algorithmically determines the magnitude of said pulse flow of gas.

6. The bidirectional incentive spirometer T-piece of claim 1 further comprising a wireless data communication device that is connected to said microprocessor; and
wherein said microprocessor stores algorithmically derived digital data for subsequent transmission.

7. The bidirectional incentive spirometer T-piece of claim 1 further comprising a hard wire data communication port that is connected to said microprocessor; and
wherein said microprocessor stores algorithmically derived digital data for subsequent transmission.

8. The bidirectional incentive spirometer T-piece of claim 1 wherein said stub and said pressure sensing unit are each configured as tapered cylinders and said pressure sensing unit frictionally engages and nests within said stub.

9. The bidirectional incentive spirometer of claim 1 wherein said proximal end port is a 15 mm ID female port and the distal end port is a 22 mm OD male port.

10. The bidirectional incentive spirometer of claim 1 wherein said distal end port is a 22 mm ID female port.
